(19) United States
(12) Patent Application Publication
(10) Pub. No.: US 2026/0198797 A1
(43) Pub. Date: Jul. 16, 2026

(54) SYSTEM FOR RESPIRATORY RATE DETERMINATIONS BASED ON MULTIPLE CONTACTLESS SENSING MODALITIES AND RELATED METHODS

(71) Applicant: KONINKLIJKE PHILIPS N.V., EINDHOVEN (NL)

(72) Inventors: SARA MARIANI, CAMBRIDGE, MA (US); DANIEL JASON SCHULMAN, JAMAICA PLAIN, MA (US); HAIBO WANG, MELROSE, MA (US); DANIEL CRAIG MCFARLANE, READING, MA (US)

(21) Appl. No.: 19/133,422

(22) PCT Filed: Dec. 4, 2023
(86) PCT No.: PCT/EP2023/084151

§ 371 Date: May 28, 2025

Related U.S. Application Data
(60) Provisional application No. 63/430,169, filed on Dec. 5, 2022.

(51) Int. Cl. A61B 5/08; A61B 5/00

(52) U.S. Cl. CPC A61B 5/0816; A61B 5/0077

(57) ABSTRACT

A system for determining a respiratory rate of a subject comprising a camera, a secondary sensor, and a controller is disclosed. The camera captures image data of the subject. The secondary sensor captures secondary sensor data corresponding to the subject. The system further includes a controller. The controller is configured to: (1) generate, via a respiratory rate model, image-based respiratory data based on the image data; (2) interpolate and/or resample the image-based respiratory data to generate an estimated respiratory waveform; (3) interpolate and/or resample the secondary sensory data to generate an estimated secondary waveform; (4) generate, via an autoregressive model, polar image-based respiratory data based on the estimated respiratory waveform; (5) generate, via the autoregressive model, polar secondary data based on the estimated secondary waveform; (6) determine a respiratory rate of the subject based on the polar image-based respiratory data and the polar secondary data.

### FIELD OF THE DISCLOSURE

The present disclosure is directed generally to respiratory rate determination based on multiple contactless sensing modalities.

### BACKGROUND

Previous advancements in the field of health monitoring have led to the development of efficient algorithms for the early prediction of infection based on laboratory values and vital signs. This technology has identified specific biomarkers most predictive of infection. Respiratory rate is one of the top predictors. Subsequent developments were aimed at models for early detection of infection outside of the hospital context, where laboratory data is typically not available, and the input to the models consisted of time series data from wearable devices.

### SUMMARY OF THE DISCLOSURE

The present disclosure furthers these advances by allowing monitoring of vital signs (heart rate, respiratory rate, temperature) for infection diagnosis and/or prediction without relying on wearable monitoring devices. This approach is particularly suited for situations in which it is inconvenient for the subject to wear a wristband, patch, or chest strap, or other type of a wearable or near-field sensor, such as in military applications where such sensors can be a burden to operations. Accordingly, state-of-the-art technology for contactless monitoring of, among other vitals signals, respiratory rate has been developed, as disclosed herein. This technology extends the detection range and improves detection at longer distances as well as in challenging situations, such as in presence of motion, multiple individuals, and different types of clothing and clothing patterns. As a result, the approach disclosed herein enables to remotely measure informative vital signs of subject in mission-critical situations at a distance with sufficient accuracy to enable early-warning determination of potential health threats that could affect readiness and performance.

The disclosed approach combines camera-based contactless monitoring of vital signs, as previously disclosed in U.S. Pat. Nos. 9,339,210 B2, 8,938,097 B2, and 9,025,826 B2, incorporated herein by reference, with distance measurements from rangefinders and audio recordings of breathing sounds using long-range microphones, such as parabolic microphones. The information from different sensors is combined using a parametric approach for spectral estimation, via autoregressive models, to improve the accuracy of the respiratory rate estimate.

The disclosed multimodal approach solves the following challenges. First, it allows remote and contactless monitoring of a vital sign (respiration rate) with improved accuracy that has high probative value for early detection of illness/infection. This is particularly important in settings where social distancing cannot be maintained, as in community living, military barracks, navy ships, etc. Also, it is important in situations where physical fitness is paramount, such as in military operations or search & rescue missions. Proper monitoring of respiratory rate is, in conjunction with other vital signs, part of a system that allows prompt and early response where otherwise infection transmission could occur.

Despite the advances in wearable technology to enhance usability and comfort, wearables such as wristbands, patches, and chest straps can be cumbersome for long term wear and in some specific subjects and settings. For example, warfighters have to wear specific equipment and operate in difficult conditions, where additional sensors can be an impediment.

Current contactless monitoring techniques can very accurately detect respiratory rate, but accuracy can be degraded at far distances (>50 m), in presence of motion, different light settings, and clothing patterns. A multimodal approach, combined with effective signal processing techniques for data fusion can mitigate this problem.

In order to implement the proposed multimodal approach, the solution disclosed herein relies on the following elements. First, a RGB camera is employed, such as, for example, a commercially available, off-the-shelf digital cameras featuring CMOS sensors and telephoto lens, such as SONY DSC-RX10 or NIKON COOLPIX P1000. Second, contactless monitoring software is used for detection of respiratory rate. Aspects of this software were previously disclosed in U.S. Pat. No. 9,265,456 B2 titled “Device and Method for Determining Vital Signs of a Subject,” issued Feb. 23, 2016, the entirety of which is incorporated by reference. Third, a rangefinder is preferably used for measuring distances. The rangefinder must be enabled to process a high frame rate and have the ability to transmit measurements to a computer. Suitable commercially available rangefinders include LEICA DISTO D810 and BOSCH GLM400CL. Fourth, a commercial, off-the-shelf parabolic microphone is used for speech recording. Example commercially available parabolic microphones include WILDTRONICS AMPLIFIED PRO MONO PARABOLIC MICROPHONE. Fifth, a computer device including a controller is employed to receive all data and runs the software for analysis and data fusion.

Generally, in one aspect, a system for determining respiratory rate of a subject is disclosed. The system includes a camera. The camera is configured to capture image data of the subject.

The system further includes a secondary sensor. The secondary sensor is configured to capture secondary sensor data corresponding to the subject. The secondary sensor may be a parabolic microphone configured to capture audio data corresponding to the subject. The secondary sensor may be a rangefinder configured to capture distance data corresponding to the subject. The camera and/or the secondary sensor may be positioned more than 50 meters from the subject.

The system further includes a controller. The controller is configured to: (1) generate, via a respiratory rate model, image-based respiratory data based on the image data; (2) interpolate and/or resample the image-based respiratory data to generate an estimated respiratory waveform; (3) interpolate and/or resample the secondary sensor data to generate an estimated secondary waveform; (4) generate, via an autoregressive model, polar image-based respiratory data based on the estimated respiratory waveform; (5) generate, via the autoregressive model, polar secondary data based on the estimated secondary waveform; (6) determine a respiratory rate of the subject based on the polar image-based respiratory data and the polar secondary data.

In some examples, the controller is further configured to filter the polar image-based respiratory data and the polar secondary data based on a breathing rate range. The breathing rate range may be 4 to 60 beats per minute.

In some examples, the controller is further configured to: (1) determining a maximum magnitude respiratory pole of the polar image-based respiratory data; and (2) determining a maximum magnitude secondary pole of the polar secondary data. The controller may determine the respiratory rate by selecting a greater magnitude value of the maximum magnitude respiratory pole and the maximum magnitude secondary pole. Alternatively, the controller may determine the respiratory rate by determining a weighted average of the maximum magnitude respiratory pole and the maximum magnitude secondary pole.

Generally, in another aspect, a method for determining respiratory rate of a subject is provided. The method includes: (1) capturing, via a camera, image data of the subject; (2) capturing, via a secondary sensor, secondary sensor data corresponding to the subject; (3) generating, via a respiratory rate model executed by a controller, image-based respiratory data based on the image data; (4) interpolating and/or resampling, via the controller, the image-based respiratory data to generate an estimated respiratory waveform; (5) interpolating and/or resampling, via the controller, the secondary sensor data to generate an estimated secondary waveform; (6) generating, via an autoregressive model executed by the controller, polar image-based respiratory data based on the estimated respiratory waveform; (7) generating, via the autoregressive model executed by the controller, polar secondary data based on the estimated secondary waveform; and (8) determining, via the controller, a respiratory rate of the subject based on the polar image-based respiratory data and the polar secondary data.

In some examples, the method may further include filtering the polar image-based respiratory data and the polar secondary data based on a breathing rate range.

In some examples, the method may further include (1) determining a maximum magnitude respiratory pole of the polar image-based respiratory data and (2) determining a maximum magnitude secondary pole of the polar secondary data. The controller may determine the respiratory rate by selecting a greater magnitude value of the maximum magnitude respiratory pole and the maximum magnitude secondary pole. Alternatively, the controller may determine the respiratory rate by determining a weighted average of the maximum magnitude respiratory pole and the maximum magnitude secondary pole.

In various implementations, a processor or controller can be associated with one or more storage media (generically referred to herein as “memory,” e.g., volatile and non-volatile computer memory such as ROM, RAM, PROM, EPROM, and EEPROM, floppy disks, compact disks, optical disks, magnetic tape, Flash, OTP-ROM, SSD, HDD, etc.). In some implementations, the storage media can be encoded with one or more programs that, when executed on one or more processors and/or controllers, perform at least some of the functions discussed herein. Various storage media can be fixed within a processor or controller or can be transportable, such that the one or more programs stored thereon can be loaded into a processor or controller so as to implement various aspects as discussed herein. The terms “program” or “computer program” are used herein in a generic sense to refer to any type of computer code (e.g., software, firmware, or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a system diagram of a system for respiratory rate determination based on multiple contactless sensing modalities, according to aspects of the present disclosure.

FIG. 2 is a respiratory waveform generated by contactless monitoring software based on image data captured by a camera, according to aspects of the present disclosure.

FIG. 3 is a distance waveform based on distance data captured by a rangefinder, according to aspects of the present disclosure.

FIG. 4 is an audio waveform based on audio data captured by a parabolic microphone, according to aspects of the present disclosure.

FIG. 5A is an interpolated and resampled respiratory waveform, according to aspects of the present disclosure.

FIG. 5B is an interpolated and resampled distance waveform compared to distance data originally captured by a rangefinder, according to aspects of the present disclosure.

FIG. 5C is a polar diagram of the respiratory waveform of FIG. 5A, according to aspects of the present disclosure.

FIG. 5D is a polar diagram of the distance waveform of FIG. 5B, according to aspects of the present disclosure.

FIG. 6 is a flowchart of a method for respiratory rate determination based on multiple contactless sensing modalities, according to aspects of the present disclosure.

### DETAILED DESCRIPTION OF EMBODIMENTS

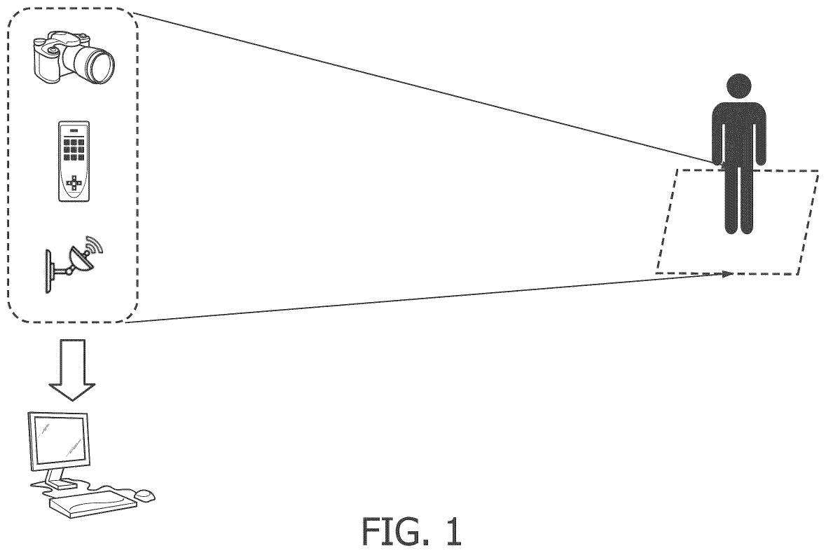

FIG. 1 is a system diagram of a system for respiratory rate determination based on multiple contactless sensing modalities. The camera, rangefinder, and microphone would be set up in a location where they can be aimed at a subject, or at a specific area that the subject could enter. Data could be collected on removable hardware (e.g., an SD card inserted in the camera), or transferred to the computer in real time.

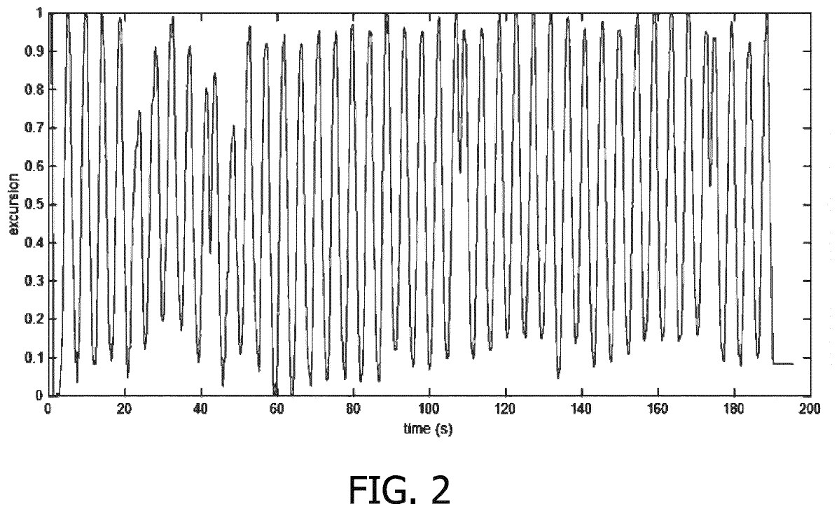

Data collection for 2 minutes allows for robust estimation of average respiratory rate in that timeframe. The camera collects video and/or images of the subject's chest area, which is then processed using contactless monitoring software. A time series representing the respiratory waveform as chest excursion is obtained, as shown in FIG. 2.

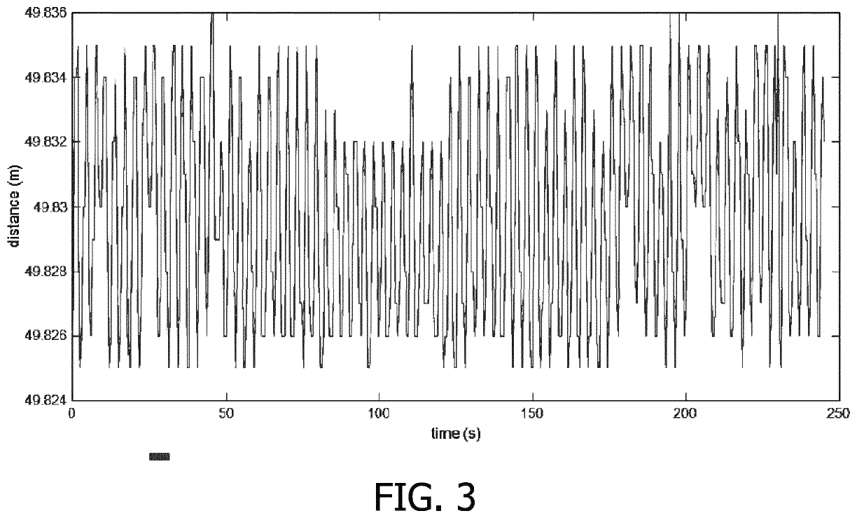

The rangefinder collects a sequence of distance measurements between the sensor and the subject's chest. With respiratory-related chest excursion, this distance changes in a cyclic fashion, and the resulting time series shows respiratory cycles, as shown in FIG. 3.

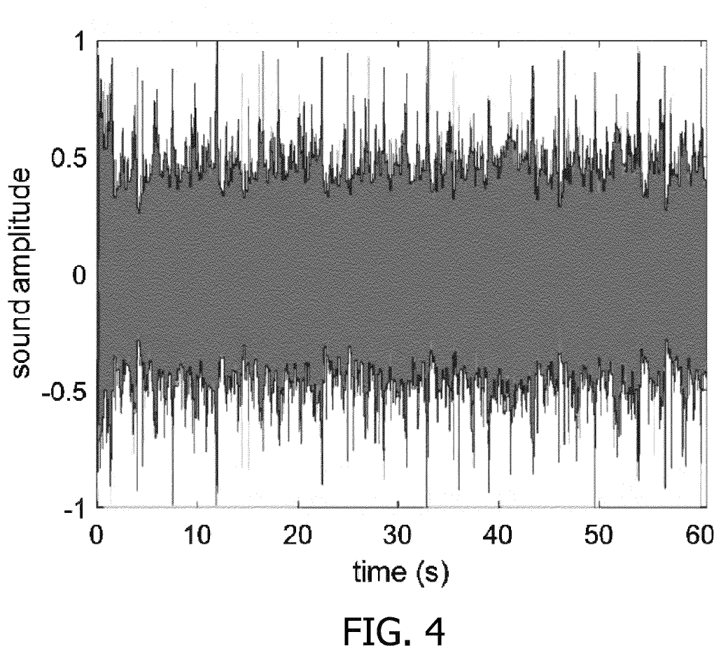

The parabolic microphone detects breathing sounds from the subject. This signal can be noisy, so filters are applied to exclude environmental noise. The resulting time series of breathing sounds is shown in FIG. 4.

The resulting time series are then processed using pole-based data fusion. Pole-based data fusion has been previously proposed for other applications, such as, for example, estimating respiratory rate from an electrocardiogram and a photoplethysmogram. This new approach combines information from multiple estimates of respiratory rate, such as, but not limited to, camera-based, rangefinder-based, and microphone-based.

For each modality, the following steps are performed: (1) interpolate the time series and resample at fixed sampling rate; (2) fit with an autoregressive (AR) model using Burg's method; (3) obtain poles of the AR model, corresponding to respiratory frequencies; (4) select poles corresponding to physiological breathing rate (such as between 4 and 60 beats per minute (bpm)); (5) for each technique, select pole with maximum magnitude; and (6) select maximum magnitude pole (or combine by weighted average (by magnitude)).

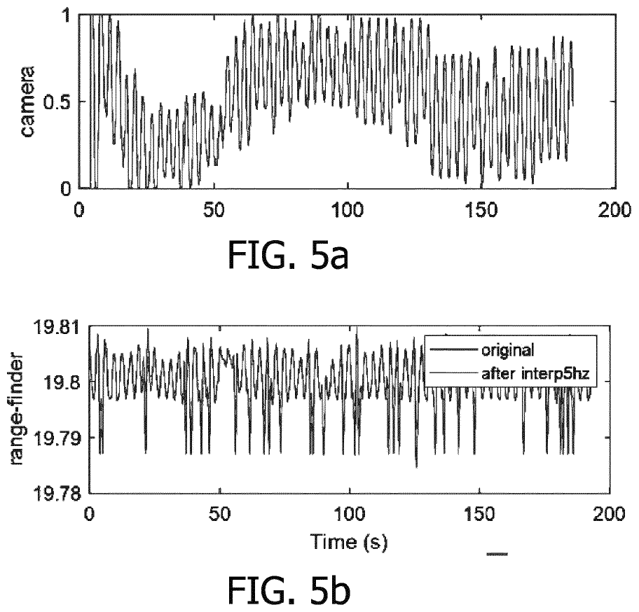

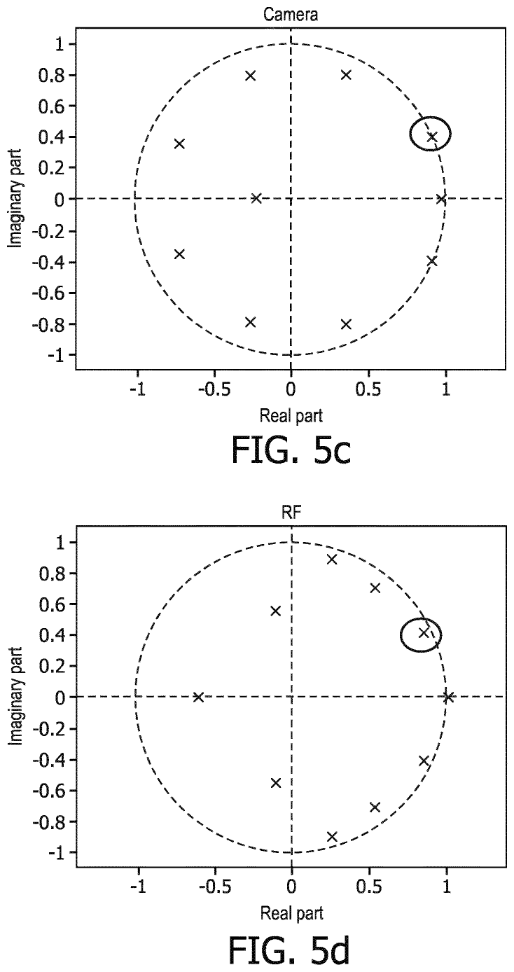

FIGS. 5A-5D represents fusion between two estimates, but it may be extended to all three estimates (image-based, distance-based, audio-based) described in this disclosure, as well as additional modalities not expressly recited herein. FIGS. 5A and 5B show the camera and rangefinder signals in the time domain after interpolation and resampling at 5 Hz. FIGS. 5C and 5D show the pole diagram for the two (camera and rangefinder) time series data sets. It can be noticed that both diagrams have a pole in the 4-60 bpm range, although the camera pole has a higher magnitude, and is thus picked by the algorithm as the respiratory rate of the subject.

This technology may have several real-world applications. One application relates to monitoring warfighters at potential risk of infection. Monitoring warfighters is important to (1) ensure their optimal performance in military operations and (2) prevent spread of pathogens in close-contact living.

Another application relates to monitoring vulnerable people in community living, e.g., elderly patients in a retirement home or inmates in a penal institution. Here, the requirement for long distance might be not as stringent, but other challenges may be in place where a multimodal approach might be desirable over a unimodal one.

Other applications relate to monitoring incoming/outcoming people in airports, train stations, and other large population centers where spread of infection can occur, for example, during a pandemic.

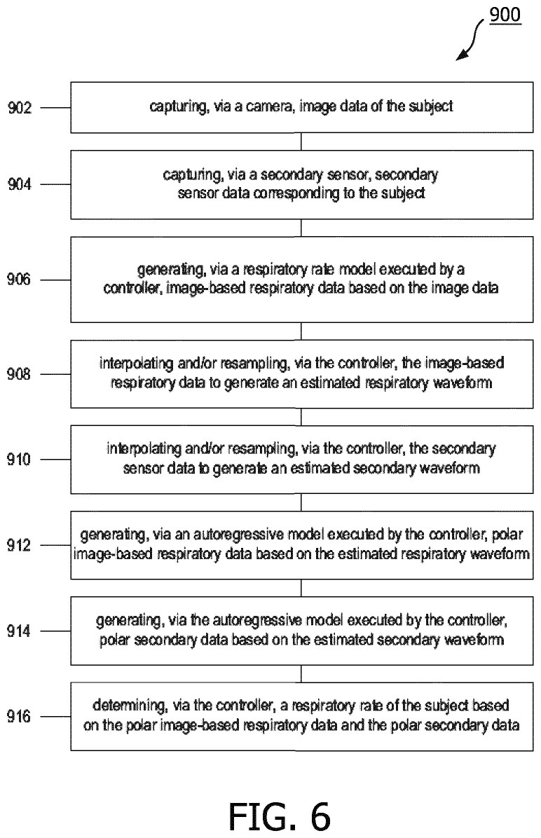

FIG. 6 is a flowchart of a method 900 for determining respiratory rate of a subject. The method includes: (1) capturing 902, via a camera, image data of the subject; (2) capturing 904, via a secondary sensor, secondary sensor data corresponding to the subject; (3) generating 906, via a respiratory rate model executed by a controller, image-based respiratory data based on the image data; (4) interpolating and/or resampling 908, via the controller, the image-based respiratory data to generate an estimated respiratory waveform; (5) interpolating and/or resampling 910, via the controller, the secondary sensor data to generate an estimated secondary waveform; (6) generating 912, via an autoregressive model executed by the controller, polar image-based respiratory data based on the estimated respiratory waveform; (7) generating 914, via the autoregressive model executed by the controller, polar secondary data based on the estimated secondary waveform; and (8) determining 916, via the controller, a respiratory rate of the subject based on the polar image-based respiratory data and the polar secondary data.

One exemplary implementation of the approach disclosed herein is described in Exhibit A attached hereto and incorporated herein by reference, reporting on an exploratory study for experimental determination of the respiratory rate of a subject, conducted by the Applicants.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects can be implemented using hardware, software, or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some examples, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific examples described herein. It is, therefore, to be understood that the foregoing examples are presented by way of example only and that, within the scope of the appended claims and equivalents thereto, examples can be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A system for determining respiratory rate of a subject, comprising:
a camera configured to capture image data of the subject;
a secondary sensor configured to capture secondary sensor data corresponding to the subject;
a controller configured to:
generate, via a respiratory rate model, image-based respiratory data based on the image data;
interpolate and/or resample the image-based respiratory data to generate an estimated respiratory waveform;
interpolate and/or resample the secondary sensor data to generate an estimated secondary waveform;
generate, via an autoregressive model, polar image-based respiratory data based on the estimated respiratory waveform;
generate, via the autoregressive model, polar secondary data based on the estimated secondary waveform;
determine a respiratory rate of the subject based on the polar image-based respiratory data and the polar secondary data.

2. The system of claim 1, wherein the secondary sensor is a parabolic microphone configured to capture audio data corresponding to the subject.

3. The system of claim 1, wherein the secondary sensor is a rangefinder configured to capture distance data corresponding to the subject.

4. The system of claim 1, wherein the controller is further configured to filter the polar image-based respiratory data and the polar secondary data based on a breathing rate range.

5. The system of claim 4, wherein the breathing rate range is 4 to 60 beats per minute.

6. The system of claim 1, wherein the controller is further configured to:
determine a maximum magnitude respiratory pole of the polar image-based respiratory data; and
determine a maximum magnitude secondary pole of the polar secondary data.

7. The system of claim 6, wherein the controller determines the respiratory rate by selecting a greater magnitude value of the maximum magnitude respiratory pole and the maximum magnitude secondary pole.

8. The system of claim 6. wherein the controller determines the respiratory rate by determining a weighted average of the maximum magnitude respiratory pole and the maximum magnitude secondary pole.

9. The system of claim 1, wherein the camera and/or the secondary sensor is positioned more than 50 meters from the subject.

10. A method for determining respiratory rate of a subject, comprising:
capturing, via a camera, image data of the subject;
capturing, via a secondary sensor, secondary sensor data corresponding to the subject;
generating, via a respiratory rate model executed by a controller, image-based respiratory data based on the image data;
interpolating and/or resampling, via the controller, the image-based respiratory data to generate an estimated respiratory waveform;
interpolating and/or resampling, via the controller, the secondary sensor data to generate an estimated secondary waveform;
generating, via an autoregressive model executed by the controller, polar image-based respiratory data based on the estimated respiratory waveform;
generating, via the autoregressive model executed by the controller, polar secondary data based on the estimated secondary waveform;
determining, via the controller, a respiratory rate of the subject based on the polar image-based respiratory data and the polar secondary data.

11. The method of claim 10, further comprising filtering the polar image-based respiratory data and the polar secondary data based on a breathing rate range.

12. The method of claim 10, further comprising:
determining a maximum magnitude respiratory pole of the polar image-based respiratory data; and
determining a maximum magnitude secondary pole of the polar secondary data.

13. The method of claim 12, wherein the controller determines the respiratory rate by selecting a greater magnitude value of the maximum magnitude respiratory pole and the maximum magnitude secondary pole.

14. The method of claim 12, wherein the controller determines the respiratory rate by determining a weighted average of the maximum magnitude respiratory pole and the maximum magnitude secondary pole.

15. The method of claim 12, wherein the secondary sensor is a parabolic microphone or a rangefinder.
